(19) United States
(12) Patent Application Publication
(10) Pub. No.: US 2024/0225887 A1
(43) Pub. Date: Jul. 11, 2024

(54) Urinal Stand Device

(71) Applicants: Charles Newman, Dilliner, PA (US); Albert Kennedy, La Plata, MD (US)

(72) Inventors: Charles Newman, Dilliner, PA (US); Albert Kennedy, La Plata, MD (US)

(21) Appl. No.: 18/398,266

(22) Filed: Dec. 28, 2023

Related U.S. Application Data
(60) Provisional application No. 63/437,390, filed on Jan. 6, 2023.

(51) Int. Cl. A61F 5/453; A61H 3/04; A61J 1/05; A61J 1/14

(52) U.S. Cl. CPC A61F 5/453; A61H 3/04; A61J 1/05; A61J 1/1462; A61H 2201/0192

(57) ABSTRACT

The urinal stand device is a specialized standing support platform to assist a disabled person to urinate without incident. The urinal stand device comprises a walker frame configured in a walker like structure, a pair of gripping handles, lower stabilization rails, and a safety plate.

When the user needs to use the bathroom, they can walk with the urinal stand device directly over the urinal or toilet due to its open front design. The open front will allow the user to get as close to the urinal or toilet as needed to avoid messes. Thus, the urinal stand device is a multiple functioning stability platform to assist with mess-free and accident-free urination for men and boys with disabilities.

### CROSS-REFERENCE TO RELATED APPLICATION

The present application claims priority to, and the benefit of, U.S. Provisional Application No. 63/437,390, which was filed on Jan. 6, 2023, and is incorporated herein by reference in its entirety.

### FIELD OF THE INVENTION

The present invention relates generally to the field of urinal stand devices. More specifically, the present invention relates to a novel way of stabilizing a male person to stand confidently at a urinal despite possible handicaps with standing. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in urinal stand devices. Generally, men and boys with disabilities and have difficulty standing, may be unable to properly utilize a urinal. Accordingly, urine can be accidentally sprayed on the urinal, walls, and floor if a shaking episode occurs in someone with a disability, requiring extensive cleaning. Consequently, being unable to independently use a urinal can be frustrating and embarrassing.

Furthermore, current walkers and stabilizing systems known in the art, do not allow for stabilization of a user when approaching and using a urinal and/or toilet. Typically, walkers use angled legs and small foot pads to allow for walking but offer less than adequate stabilization while standing in small places.

Accordingly, there is a demand for an improved urinal stand device that provides users with a stable walker for use while standing at a urinal. The urinal stand device allows men and boys with disabilities to independently stand and utilize a urinal without a mess or accidents occurring.

Therefore, there exists a long-felt need in the art for a urinal stand device that provides a stable walker for use while standing at a urinal for boys and men with disabilities. There is also a long-felt need in the art for a urinal stand device that prevents loss of balance and provides stable holding handles for a more comfortable and steady experience. Further, there is a long-felt need in the art for a urinal stand device that decreases the occurrence of urine splashing and other accidents. Moreover, there is a long-felt need in the art for a urinal stand device that has stabilizing plates and rails along the lower portion of the device for a lower center of gravity and more stable foundation. Further, there is a long-felt need in the art for a urinal stand device that incorporates a standing, safety plate that helps to prevent the device from moving when not wanted. Finally, there is a long-felt need in the art for a urinal stand device that is designed to work with any urinal or toilet to allow for use across any bathroom situation.

The subject matter disclosed and claimed herein, in one embodiment thereof, comprises a urinal stand device. The device is a specialized standing support platform to assist in the ability of a disabled person to urinate without incident. The urinal stand device comprises a walker frame configured in a walker-like structure with a plurality of horizontal members and vertical legs. The walker frame is configured in an open-front design with a pair of rear vertical legs positioned a distance apart and being of a length adjustable to a user's height. A pair of front vertical legs are positioned lower than the pair of rear vertical legs. A horizontal member connects the pair of rear vertical legs at the bottom, middle, and top, as needed. Further horizontal members connect the pair of rear vertical legs to the pair of front vertical legs, leaving the pair of front vertical legs open and able to encompass a toilet or urinal. The open front of the walker frame will allow the user to get as close to the urinal or toilet, as needed.

The walker frame also comprises a pair of gripping handles, lower stabilization rails, and a safety plate. The lower stabilization rails secure the front and rear vertical legs together where the device meets the ground to add stability. Further, the pair of gripping handles are placed in a position to allow for any size user and to keep the user at the proper distance from the urinal or toilet. The safety plate adds stability and a step for users, if needed. The urinal stand device can also incorporate a portable urinal if needed, when no permanent urinal or toilet is available. When the user needs to use the bathroom, they can walk with the urinal stand device to the urinal or toilet. Thus, the urinal stand device is a multiple functioning stability platform to assist with mess-free and accident-free urination for men and boys with disabilities.

In this manner, the urinal stand device of the present invention accomplishes all of the foregoing objectives and provides users with a device that prevents accidents or mess when a disabled person needs to use a toilet or urinal. The urinal stand device allows a user to approach any toilet or urinal and never lose grip of the handles. By allowing the urinal stand device to position as close to a urinal or toilet as needed, there is less room for mess or accidents.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a urinal stand device. The device is a specialized standing support platform to assist in the ability of a disabled person to urinate without incident. The urinal stand device comprises a walker frame configured in a walker-like structure. The walker frame is configured in an open-front design that allows the user to get as close to the urinal or toilet, as needed. Accordingly, when the user needs to use the bathroom, they can walk with the urinal stand device to the urinal or toilet. Thus, the urinal stand device is a multiple functioning stability platform to assist with mess-free and accident-free urination for men and boys with disabilities.

In one embodiment, the urinal stand device is described as a walker-like structure. The term “walker” as used herein, broadly refers to a device that assists a user when walking by providing a platform upon which the user can lean and steady their balance. In some embodiments, the walker can be configured as a rollator, which enables the user to walk with the support of a frame and wheels.

In one embodiment, the urinal stand device comprises a walker frame upon which a user can lean to obtain upper body support while urinating. It should be appreciated that walker frame can have any desired configuration that can support a user while urinating into a toilet or urinal, as described below. As shown, the walker frame includes a pair of front vertical legs and a pair of rear vertical legs, wherein the front vertical legs are positioned lower than the rear vertical legs. Further, the length of the front and rear vertical legs can be adjusted using any known mechanism to accommodate users of different heights, as long as the front vertical legs are positioned consistently lower than the rear vertical legs. For example, in some embodiments, each vertical leg can include a telescoping feature to allow for lengthening and shortening. However, any known method of adjusting the length of the legs can be used as is known in the art.

In one embodiment, a horizontal member connects the rear vertical legs at a bottom, middle, and/or top, as needed. The bottom horizontal member can be stepped on if needed, to prevent the loss of balance while holding the handles and urinating. Further horizontal members connect the front vertical legs to the rear vertical legs at the top and middle, if necessary. Any suitable number of horizontal members can be utilized to connect the vertical legs, as is known in the art, to add stability and support. The horizontal members are connected together via any suitable securing means, as is known in the art, such as spring-buttons, screws, welds, etc. Additionally, the front vertical legs are not connected together with a horizontal member, leaving the front vertical legs open and able to encompass a toilet or urinal. Thus, the open front of the walker frame will allow the user to get as close to the urinal or toilet, as needed.

In one embodiment, the walker frame comprises a pair of lower stabilization rails that secure the front and rear vertical legs together. The lower stabilization rails are connected where the device meets the ground to add stability. The lower stabilization rails are typically wider in size and shape compared to the horizontal members, thus creating a stand and allowing for more stability for the walker frame when resting upright over a toilet or urinal.

In one embodiment, the walker frame can include a pair of front wheels and/or a pair of rear wheels attached to the bottom surface of each vertical leg. The wheels can have any desired configuration, such as an inflated tire, a solid rubber material, or a plastic sphere. In some embodiments, the wheels are connected with a swivel device (i.e., caster-mounted) to enable the user to move in any direction immediately. In other embodiments, only front wheels are configured to swivel, while rear wheels are affixed in a forward-reverse direction. It should be appreciated that the presently disclosed subject matter also includes embodiments wherein the walker is configured with two wheels or without wheels.

In one embodiment, the walker frame can include an optional receptacle to house and protect a user's belongings (i.e., keys, glasses, books, snacks, etc.). The receptacle can have any desired configuration such as (but not limited to) a tray, basket, drawer, shelf, and the like. In some embodiments, the receptacle is removable. In other embodiments, the receptacle is permanently mounted to the walker frame.

In one embodiment, the walker frame comprises a pair of handles supported by the frame, that provide a surface for the user to grasp when using the urinal stand device. The handles extend out from the rear vertical legs. The handles provide a gripping surface, allowing the user to steer the device by pushing or pulling on the handles, as needed. The handles can also aid in maintaining balance of the user when urinating or standing. The height and/or angle of each handle can be adjusted using any known mechanism, to allow for any size user. For example, a user-accessible telescoping feature can be used to adjust each handle, and a pushpin can be used to selectively lock the handle at a desired position. The hand grip portion of each handle can be straight or ergonomically designed. Further, the hand grip can be constructed from one or more non-slip materials, such as rubber, plastic, and the like.

In one embodiment, each handle can include a brake positioned directly beneath for ease of use. However, the brake can be housed in any location. The brake allows the user to have control over their speed, positioning, and/or preventing the wheels from rolling. In some embodiments, the brake is controlled through a cable running down the side of walker frame and coordinating with rear wheels. Grasping the brake can cause the cable to pull up on a pivoting member that causes a brake element to come into frictional contact with a corresponding wheel. The hand braking mechanism is well known in the art. It should be appreciated that the presently disclosed subject matter is not limited and any braking mechanism can be used to lock one or more wheels in place.

In some embodiments, each brake handle is independently operable and connected to a brake apparatus. In this way, the user can stop the walker using one or both hands.

In one embodiment, the walker frame comprises a safety plate which can be added for further weight accommodation and stability. The safety plate can be manufactured of any desired material, such as aluminum, plastic, wood, or can be made of the same material as the walker frame. The safety plate is easy to install and remove from the walker frame. Typically, the safety plate comprises the dimensions of 19.5 inches by 12 inches by 0.25 inches thick, or any other suitable dimensions as is known in the art. Further, when the user steps on the safety plate, the weight of the device increases from approximately 13 pounds to 13 pounds plus the weight of the user, thus, making the device more stable. In one embodiment, the safety plate could be stepped on by the user to help prevent loss of balance while holding the handles and urinating.

In one embodiment, the walker frame comprises a urinal bottle that can be hung from one of the horizontal members, as needed. Thus, if a user cannot get to a physical toilet or urinal in time, the urinal bottle can be used. Further, the urinal stand device can also incorporate any suitable portable urinal as is known in the art, when no permanent urinal or toilet is available.

Although not shown, it should be appreciated that additional accessories can be easily incorporated into the walker frame. For example, optional footrests, drink holders, and/or arm cushions can be added, as needed.

In one embodiment, the walker frame can be constructed from any desired material. For example, suitable materials can include (but are not limited to) metal (i.e., steel, aluminum, titanium, stainless steel), plastic, carbon fiber, wood, or combinations thereof. The materials used to construct the walker frame should be able to withstand the weight of the user, yet still remain lightweight for transportation.

To accommodate ease of transport, the walker frame can be configured to fold about pivot points between an unfolded state and a folded state. In this way, the walker frame can be folded to a compact state to better facilitate storing the walker frame in a closet, under a bed, or behind a door. However, the presently disclosed subject matter is not limited and includes embodiments wherein the use position does not fold to a storage position.

In yet another embodiment, the urinal stand device comprises a plurality of indicia.

In yet another embodiment, a method of assisting disabled users in utilizing a toilet or urinal is disclosed. The method includes the steps of providing a urinal stand device comprising a walker frame configured in a walker-like structure, with lower stabilization rails, a pair of handles, and a safety plate. The method also comprises walking with the device to a toilet or urinal. Further, the method comprises engaging the open front area of the walker frame with the urinal or toilet. The method also comprises stepping up onto the safety plate, as needed. Finally, the method comprises using the toilet or urinal to urinate while being stabilized by the device.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the urinal stand device of the present invention showing a user walking with the device in accordance with the disclosed architecture;

FIG. 2 illustrates a perspective view of one embodiment of the urinal stand device of the present invention showing the stabilizing rails in accordance with the disclosed architecture;

FIG. 3 illustrates a perspective view of one embodiment of the urinal stand device of the present invention showing the safety plate in accordance with the disclosed architecture;

FIG. 4 illustrates a perspective view of one embodiment of the urinal stand device of the present invention showing how the device allows for positioning around a toilet in accordance with the disclosed architecture; and

FIG. 5 illustrates a flowchart showing the method of assisting disabled users in utilizing a toilet or urinal in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a urinal stand device that provides users with a stable walker for use while standing at a urinal for boys and men with disabilities. There is also a long-felt need in the art for a urinal stand device that prevents loss of balance and provides stable holding handles for a more comfortable and steady experience. Further, there is a long-felt need in the art for a urinal stand device that decreases the occurrence of urine splashing and other accidents. Moreover, there is a long-felt need in the art for a urinal stand device that has stabilizing plates and rails along the lower portion of the device for a lower center of gravity and more stable foundation. Further, there is a long-felt need in the art for a urinal stand device that incorporates a standing, safety plate that helps to prevent the device from moving when not wanted. Finally, there is a long-felt need in the art for a urinal stand device that is designed to work with any urinal or toilet to allow for use across any bathroom situation.

The present invention, in one exemplary embodiment, is a novel urinal stand device. The device is a specialized standing support platform to assist in the ability of a disabled person to urinate without incident. The urinal stand device comprises a walker frame configured in a walker-like structure with a plurality of horizontal members and vertical legs. The walker frame is configured in an open-front design with a pair of rear vertical legs positioned a distance apart and being of a length adjustable to a user's height. A pair of front vertical legs are positioned lower than the pair of rear vertical legs. A horizontal member connects the pair of rear vertical legs at the bottom, middle, and top, as needed. Further horizontal members connect the pair of rear vertical legs to the pair of front vertical legs, leaving the pair of front vertical legs open and able to encompass a toilet or urinal. The open front of the walker frame will allow the user to get as close to the urinal or toilet as needed. The present invention also includes a novel method of assisting disabled users in utilizing a toilet or urinal. The method includes the steps of providing a urinal stand device comprising a walker frame configured in a walker-like structure, with lower stabilization rails, a pair of handles, and a safety plate. The method also comprises walking with the device to a toilet or urinal. Further, the method comprises engaging the open front area of the walker frame with the urinal or toilet. The method also comprises stepping up onto the safety plate, as needed. Finally, the method comprises using the toilet or urinal to urinate while being stabilized by the device.

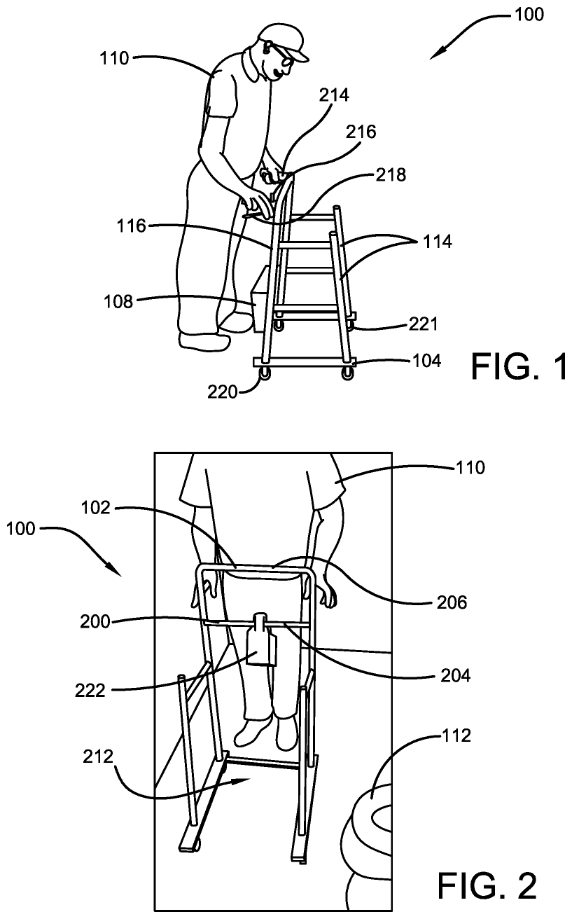

Referring initially to the drawings, FIG. 1 illustrates a perspective view of one embodiment of the urinal stand device 100 of the present invention. In the present embodiment, the urinal stand device 100 is an improved urinal stand device 100 that is a device for assisting disabled men and boys urinate while standing up. Specifically, the urinal stand device 100 comprises a walker frame 102 configured in a walker-like structure, with lower stabilization rails 104, a pair of handles 106, and a safety plate 108. The walker frame 102 is configured in an open-front design that allows the user 110 to get as close to the urinal or toilet 112 as needed. Accordingly, when the user 110 needs to use the bathroom, they can walk with the urinal stand device 100 to the urinal or toilet 112. Thus, the urinal stand device 100 is a multiple functioning stability platform to assist with mess-free and accident-free urination for men and boys with disabilities.

Generally, the urinal stand device 100 is described as a walker-like structure. The term “walker” as used herein, broadly refers to a device that assists a user 110 when walking, standing, or urinating by providing a platform upon which the user 110 can lean and steady their balance. In some embodiments, the walker-like structure can be configured as a rollator, which enables the user 110 to walk with the support of a frame and wheels.

Furthermore, the urinal stand device 100 comprises a walker frame 102 upon which a user 110 can lean to obtain upper body support while urinating. It should be appreciated that the walker frame 102 can have any desired configuration that can support a user 110 while urinating into a toilet or urinal 112, as described below. As shown, the walker frame 102 includes a pair of front vertical legs 114 and a pair of rear vertical legs 116, wherein the front vertical legs 114 are positioned lower than the rear vertical legs 116. Specifically, the front vertical legs 114 are positioned closest to the toilet 112 and the rear vertical legs 116 are positioned closest to the user 110.

Further, the length of the front 114 and rear 116 vertical legs can be adjusted using any known mechanism to accommodate users 110 of different heights, as long as the front vertical legs 114 are positioned consistently lower than the rear vertical legs 116. For example, in some embodiments, each vertical leg 114 and 116 can include a telescoping feature to allow for lengthening and shortening. However, any known method of adjusting the length of the legs 114 and 116 can be used as is known in the art.

As shown in FIG. 2, a horizontal member 200 connects the rear vertical legs 116 at the bottom 202, middle 204, and/or top 206, as needed. The bottom horizontal member 206 can be stepped on if needed, to prevent the loss of balance while holding the handles 106 and urinating. Further horizontal members 200 connect the front vertical legs 114 to the rear vertical legs 116 at the top 208 and middle 210, if necessary. Any suitable number of horizontal members 200 can be utilized to connect the vertical legs 114 and 116, as is known in the art, to add stability and support. The horizontal members 200 are connected together via any suitable securing means, as is known in the art, such as spring-buttons, screws, welds, etc. Additionally, the front vertical legs 114 are not connected together with a horizontal member 200, leaving the front vertical legs 114 open and able to encompass a toilet or urinal 112. Thus, the open front 212 of the walker frame 102 will allow the user 110 to get as close to the urinal or toilet 112, as needed.

Further, the walker frame 102 comprises a pair of lower stabilization rails 104 that secure the front 114 and rear 116 vertical legs together. The lower stabilization rails 104 are connected where the device 100 meets the ground to add stability. The lower stabilization rails 104 are typically wider in size and shape compared to the horizontal members 200, thus creating a stand and allowing for more stability for the walker frame 102 when resting upright over a toilet or urinal 112.

Additionally, the walker frame 102 comprises a pair of handles 106 supported by the frame 102, that provide a surface for the user 110 to grasp when using the urinal stand device 100. The handles 106 extend out from the rear vertical legs 116. The handles 106 provide a gripping surface 214, allowing the user 110 to steer the device 100 by pushing or pulling on the handles 106 as needed. The handles 106 can also aid in maintaining balance of the user 110 when urinating or standing. The height and/or angle of each handle 106 can be adjusted using any known mechanism, to allow for any size user 110. For example, a user-accessible telescoping feature can be used to adjust each handle 106, and a pushpin 216 can be used to selectively lock the handle 106 at a desired position. The hand grip portion 214 of each handle 106 can be straight or ergonomically designed. Further, the hand grip 214 can be constructed from one or more non-slip materials, such as rubber, plastic, and the like.

In one embodiment, each handle 106 can include a brake 218 positioned directly beneath for ease of use. However, the brake 218 can be housed in any location. The brake 218 allows the user 110 to have control over their speed, positioning, and/or preventing the wheels 220 from rolling. In some embodiments, the brake 218 is controlled through a cable running down the side of walker frame 102 and coordinating with rear wheels 220. Grasping the brake 218 can cause the cable to pull up on a pivoting member that causes a brake element to come into frictional contact with a corresponding wheel 220. The hand braking mechanism is well known in the art. It should be appreciated that the presently disclosed subject matter is not limited and any braking mechanism can be used to lock one or more wheels 220 in place.

In some embodiments, each brake handle 218 is independently operable and connected to a brake apparatus. In this way, the user 110 can stop the device 100 using one or both hands.

In one embodiment, the walker frame 102 comprises a urinal bottle 222 that can be hung from one of the horizontal members 200, as needed. Thus, if a user 110 cannot get to a physical toilet or urinal 112 in time, the urinal bottle 222 can be used. Further, the urinal stand device 100 can also incorporate any suitable portable urinal bottle 222 as is known in the art, when no permanent urinal or toilet 112 is available.

To accommodate ease of transport, the walker frame 102 can be configured to fold about pivot points 224 between an unfolded state and a folded state. In this way, the walker frame 102 can be folded to a compact state to better facilitate storing the walker frame 102 in a closet, under a bed, or behind a door. However, the presently disclosed subject matter is not limited and includes embodiments wherein the use position does not fold to a storage position.

As shown in FIG. 3, in one embodiment, the walker frame 102 can include a pair of front wheels 221 and/or a pair of rear wheels 220 attached to the bottom surface of each vertical leg 114 and 116. The wheels 220 can have any desired configuration, such as an inflated tire, a solid rubber material, or a plastic sphere. In some embodiments, the wheels 220 are connected with a swivel device (i.e., caster-mounted) to enable the user 110 to move in any direction immediately. In other embodiments, only front wheels 221 are configured to swivel, while rear wheels 220 are affixed in a forward-reverse direction. It should be appreciated that the presently disclosed subject matter also includes embodiments wherein the device 100 is configured with two wheels 220 or without wheels 220.

In one embodiment, the walker frame 102 can include an optional receptacle 226 to house and protect a user's belongings (i.e., keys, glasses, books, snacks, etc.). The receptacle 226 can have any desired configuration such as (but not limited to) a tray, basket, drawer, shelf, and the like. In some embodiments, the receptacle 226 is removable. In other embodiments, the receptacle 226 is permanently mounted to the walker frame 102.

In another embodiment, the walker frame 102 comprises a safety plate 108 which can be added for further weight accommodation and stability. The safety plate 108 can be manufactured of any desired material, such as aluminum, plastic, wood, or can be made of the same material as the walker frame 102. The safety plate 108 is easy to install and remove from the walker frame 102. Typically, the safety plate 108 comprises the dimensions of 19.5 inches by 12 inches by 0.25 inches thick, or any other suitable dimensions as is known in the art. Further, when the user 110 steps on the safety plate 108, the weight of the device 100 increases from approximately 13 pounds to 13 pounds plus the weight of the user 110, thus, making the device 100 more stable. In one embodiment, the safety plate 108 could be stepped on by the user 110 to help prevent loss of balance while holding the handles 106 and urinating.

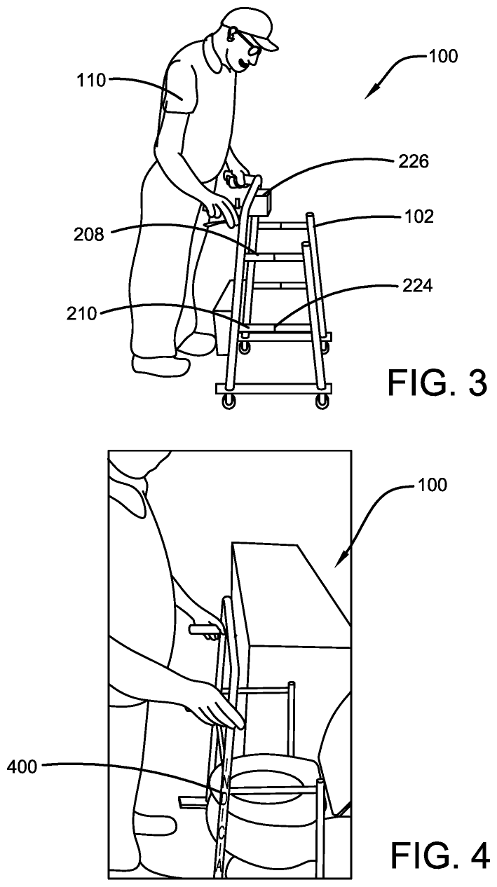

As shown in FIG. 4, the walker frame 102 can be constructed from any desired material. For example, suitable materials can include (but are not limited to) metal (i.e., steel, aluminum, titanium, stainless steel), plastic, carbon fiber, wood, or combinations thereof. The materials used to construct the walker frame 102 should be able to withstand the weight of the user 110, yet still remain lightweight for transportation.

Although not shown, it should be appreciated that additional accessories can be easily incorporated into walker frame 102. For example, optional footrests, drink holders, and/or arm cushions can be added, as needed.

In yet another embodiment, the urinal stand device 100 comprises a plurality of indicia 400. The walker frame 102 of the device 100 may include advertising, a trademark, or other letters, designs, or characters, printed, painted, stamped, or integrated into the walker frame 102, or any other indicia 400 as is known in the art. Specifically, any suitable indicia 400 as is known in the art can be included, such as, but not limited to, patterns, logos, emblems, images, symbols, designs, letters, words, characters, animals, advertisements, brands, etc., that may or may not be walker, toilet, urinal, or brand related.

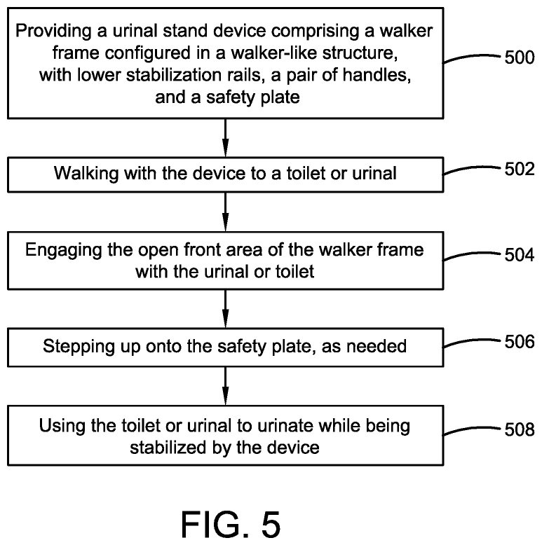

FIG. 5 illustrates a flowchart of the method of assisting disabled users in utilizing a toilet or urinal. The method includes the steps of at 500, providing a urinal stand device comprising a walker frame configured in a walker-like structure, with lower stabilization rails, a pair of handles, and a safety plate. The method also comprises at 502, walking with the device to a toilet or urinal. Further, the method comprises at 504, engaging the open front area of the walker frame with the urinal or toilet. The method also comprises at 506, stepping up onto the safety plate, as needed. Finally, the method comprises at 508, using the toilet or urinal to urinate while being stabilized by the device.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “urinal stand device”, “urinal device”, and “device” are interchangeable and refer to the urinal stand device 100 of the present invention.

Notwithstanding the foregoing, the urinal stand device 100 of the present invention can be of any suitable size and configuration as is known in the art without affecting the overall concept of the invention, provided that it accomplishes the above-stated objectives. One of ordinary skill in the art will appreciate that the urinal stand device 100 as shown in FIGS. 1-5 is for illustrative purposes only, and that many other sizes and shapes of the urinal stand device 100 are well within the scope of the present disclosure. Although the dimensions of the urinal stand device 100 are important design parameters for user convenience, the urinal stand device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An urinal stand device that assists disabled men and boys in urinating while standing up, the urinal stand device comprising:
a walker frame;
a pair of handles; and
lower stabilization rails;
wherein the pair of handles are secured to the walker frame and allow a user to hold the handles for support;
wherein the walker frame is configured in an open-front design that allows a user to get as close to the urinal or toilet, as needed; and
further wherein the lower stabilization rails are secured to a bottom of the walker frame to provide support for the walker frame when in an upright position.

2. The urinal stand device of claim 1, wherein the walker frame comprises a pair of front vertical legs and a pair of rear vertical legs, wherein the front vertical legs are positioned shorter than the rear vertical legs.

3. The urinal stand device of claim 2, wherein the front vertical legs are positioned closest to the toilet or urinal and the rear vertical legs are positioned closest to the user.

4. The urinal stand device of claim 3, wherein length of the front and rear vertical legs can be adjusted using a telescoping feature.

5. The urinal stand device of claim 4, wherein a horizontal member connects the rear vertical legs at a bottom position, a middle position, and a top position.

6. The urinal stand device of claim 5, wherein the bottom horizontal member can be stepped on if needed, to prevent loss of balance while holding the pair of handles and urinating.

7. The urinal stand device of claim 6, wherein horizontal members connect the front vertical legs to the rear vertical legs at a top and a middle position.

8. The urinal stand device of claim 7, wherein the front vertical legs are not connected together with a horizontal member, leaving the front vertical legs open and able to encompass the toilet or urinal.

9. The urinal stand device of claim 8, wherein the lower stabilization rails secure the front and rear vertical legs together, are wider in size and shape compared to the horizontal members, and are connected where the urinal stand device meets a ground surface to add stability.

10. The urinal stand device of claim 9, wherein the pair of handles extend out from the rear vertical legs.

11. The urinal stand device of claim 10, wherein the pair of handles comprise a hand grip portion constructed of non-slip materials.

12. The urinal stand device of claim 11, wherein a bottom of the walker frame comprises a safety plate which can be added for further weight accommodation and stability.

13. The urinal stand device of claim 12 further comprising a urinal bottle that can be hung from one of the horizontal members, as needed.

14. An urinal stand device that assists disabled men and boys in urinating while standing up, the urinal stand device comprising:
a walker frame comprising a pair of front vertical legs and a pair of rear vertical legs, wherein the front vertical legs are positioned shorter than the rear vertical legs;
a pair of handles that extend out from the rear vertical legs; and
lower stabilization rails which secure the front and rear vertical legs together, are wider in size and shape compared to horizontal members, and are connected where the urinal stand device meets a ground surface to add stability; and
wherein the pair of handles are secured to the walker frame and allow a user to hold the handles for support;
wherein the front vertical legs are positioned closest to the toilet or urinal and the rear vertical legs are positioned closest to the user;
wherein length of the front and rear vertical legs can be adjusted using a telescoping feature;
wherein a horizontal member connects the rear vertical legs at a bottom position, a middle position, and a top position;
wherein the bottom horizontal member can be stepped on if needed, to prevent loss of balance while holding the pair of handles and urinating;
wherein horizontal members connect the front vertical legs to the rear vertical legs at a top and a middle position;
wherein the front vertical legs are not connected together with a horizontal member, leaving the front vertical legs open and able to encompass the toilet or urinal;
wherein the walker frame is configured in an open-front design that allows a user to get as close to the urinal or toilet, as needed;
wherein a bottom of the walker frame comprises a safety plate which can be added for further weight accommodation and stability; and
further wherein a urinal bottle can be hung from one of the horizontal members, as needed.

15. The urinal stand device of claim 14, wherein the walker frame can be configured to fold about pivot points between an unfolded state and a folded state.

16. The urinal stand device of claim 14 further comprising wheels attached to a bottom surface of each vertical leg.

17. The urinal stand device of claim 14, wherein each handle includes a brake positioned directly beneath for ease of use.

18. The urinal stand device of claim 14, wherein the walker frame comprises an optional receptacle to house and protect a user's belongings.

19. The urinal stand device of claim 14 further comprising a plurality of indicia.

20. A method of assisting disabled users in utilizing a toilet or urinal, the method comprising the following steps:
providing a urinal stand device comprising a walker frame configured in a walker-like structure, with lower stabilization rails, a pair of handles, and a safety plate;
walking with the urinal stand device to a toilet or urinal;
engaging the open front area of the walker frame with the urinal or toilet;
stepping up onto the safety plate, as needed; and
using the toilet or urinal to urinate while being stabilized by the urinal stand device.
